(19) United States
(12) Patent Application Publication
(10) Pub. No.: US 2007/0270940 A1
(43) Pub. Date: Nov. 22, 2007

(54) Bioabsorbable Magnesium-Reinforced Polymer Stents

(75) Inventor: David Doty, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular. Inc., Santa Rosa, CA (US)

(21) Appl. No.: 11/744,977

(22) Filed: May 7, 2007

Related U.S. Application Data
(60) Provisional application No. 60/747,389, filed on May 16, 2006.

(51) Int. Cl. A61F 2/88

(52) U.S. Cl. 623/1.22; 623/1.38

(57) ABSTRACT

Bioabsorbable magnesium-reinforced polymer stents are disclosed. Additionally, bioabsorbable magnesium-reinforced polymer stents are disclosed which elute therapeutic agents.

### RELATED APPLICATIONS

This application claims the benefit of U.S. Provisional Patent Application 60/747,389 filed May 16, 2006.

### FIELD OF THE INVENTION

The present invention relates to bioabsorbable magnesium-reinforced stents. More specifically, the present invention provides bioabsorbable polymeric vascular stents reinforced with bioabsorbable magnesium alloys.

### BACKGROUND OF THE INVENTION

Implantable medical devices have become increasingly more common over the last 50 years and have found applications in nearly every branch of medicine. Examples include joint replacements, vascular grafts, heart valves, ocular lenses, pacemakers, vascular stents, urethral stents, and many others. Regardless of the application, however, implantable medical devices must be biocompatible, that is, they must be fabricated from materials that will not elicit an adverse biological response such as, but not limited to, inflammation, thrombogenesis or necrosis. Early medical devices were generally fabricated from inert materials such as precious metals and ceramics. More recently, stainless steel and other metal alloys have replaced precious metals and polymers are also being substituted for ceramics.

Generally, implantable medical devices are intended to serve long term therapeutic applications and are not removed once implanted. In some cases it may be desirable to use implantable medical devices for short term therapies. Their removal, however, may require highly invasive surgical procedures that place the patient at risk for life threatening complications. It would be desirable to have medical devices designed for short term applications that degrade via normal metabolic pathways and are reabsorbed into the surrounding tissues.

One of the first bioresorbable medical devices developed was the synthetic absorbable suture marketed as Dexon in the 1960s by Davis and Geck, Inc. (Danbury, Conn.). Since that time, diverse biodegradable polymer-based products have found acceptance as implantable medical devices and implantable medical device coatings, thereby alleviating the need for secondary invasive procedure(s) to remove implanted medical device(s).

Additionally, recent advances in in situ drug delivery have led to the development of implantable medical devices specifically designed to provide therapeutic compositions to remote anatomical locations. Perhaps one of the most exciting areas of in situ drug delivery is in the field of interventional cardiology. Vascular occlusions leading to ischemic heart disease are frequently treated using percutaneous transluminal coronary angioplasty (PTCA) whereby a dilation catheter is inserted through a femoral artery incision and directed to the site of the vascular occlusion. The catheter is dilated and the expanding catheter tip (the balloon) opens the occluded artery restoring vascular patency. Generally, a vascular stent is deployed at the treatment site to minimize vascular recoil and restenosis. In some cases, however, stent deployment leads to damage to the intimal lining of the artery which may result in vascular smooth muscle cell hyperproliferation and restenosis. When restenosis occurs it is necessary to either re-dilate the artery at the treatment site, or, if that is not possible, a surgical coronary artery bypass procedure must be performed.

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMCs proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

The introduction of intracoronary stents into clinical practice has dramatically changed treatment of obstructive coronary artery disease. Since having been shown to significantly reduce restenosis as compared to percutaneous transluminal coronary angioplasty (PTCA) in selected lesions, the indication for stent implantation has been widened substantially. As a result of a dramatic increase in implantation numbers worldwide in less selected and more complex lesions, in-stent restenosis (ISR) has been identified as a new medical problem with significant clinical and socioeconomic implications. The number of ISR cases is growing: from 100,000 patients treated worldwide in 1997 to an estimated 150,000 cases in 2001 in the United States alone. ISR is due to a vascular response to injury, and this response begins with endothelial denudation and culminates in vascular remodeling after a significant phase of smooth muscle cell proliferation.

Stents, useful for restoring and maintaining patency in biological lumens, can be manufactured from a variety of materials. These materials include, but are not limited to, metals and polymers. Both metal and polymer vascular stents have been associated with thrombosis and chronic inflammation at the implantation site and impaired remodeling at the stent site. It has been proposed that limiting the exposure of the vessel to the stent to the immediate intervention period would reduce late thrombosis and chronic inflammation. One means to produce a temporary stent is to implant a bioabsorbable, or biodegradable, stent.

There are several parameters to consider in the selection of a bioabsorbable material for stent manufacture. These include, but are not limited to, the strength of the polymer to avoid potential immediate recoil, the rate of degradation and corrosion, biocompatibility with the vessel wall and lack of toxicity. Additionally, it may be desirable to include therapeutic agents in the bioabsorbable stent such that the therapeutic agent is release at the implantation site during degradation of the stent. The mechanical properties and release profiles of therapeutic agents directly depend on the rate of degradation of the stent material which is controlled by selection of the stent materials, passivation agents and the manufacturing process of the stent. Currently there are two types of materials used in bioabsorbable stents, polymers and metals.

Bioabsorbable polymer stent materials have several significant limitations. Their radial strength is lower than metallic stents which can result in early recoil postimplantation, they are associated with a significant degree of local inflammation, their bioabsorption rate can be relatively slow, and they may still result in restenosis. Additional polymeric stent are often radiolucent which impairs accurate positioning within a vessel lumen. The physical limitations of the polymer require thick struts to increase radial strength which impedes their profile and delivery capabilities, especially in small vessels.

Metal bioabsorbable stents are attractive since they have the potential to perform similarly to stainless steel metal stents. One such material is magnesium and bioresorbable magnesium alloy stents have been shown to induce less thrombosis in damaged arteries than conventional bare metal stents.

Therefore, there exists a need for a bioabsorbable stent material which incorporates the strength characteristics of a metal with the drug eluting properties of a polymer.

### SUMMARY OF THE INVENTION

The present invention provides bioabsorbable magnesium-reinforced polymer stents which combine the radial strength and flexibility of metal stents with the controlled drug delivery properties of polymers.

In one embodiment of the present invention, a stent is provided comprising a bioabsorbable magnesium-reinforced polymer.

In another embodiment of the present invention, the bioabsorbable magnesium comprises magnesium and magnesium alloys. In another embodiment, the magnesium alloy comprises an alloy of magnesium, aluminum and zinc.

In another embodiment, the bioabsorbable polymer is selected from the group consisting of polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations thereof.

In yet another embodiment of the present invention, the stent is selected from the group consisting of woven stents, individual ring stents, sequential ring stents, closed cell stents, open cell stents, laser cut tube stents, ratcheting stents, and modular stents. In another embodiment, the stent is a vascular stent. In yet another embodiment, the stent is a helical spiral vascular stent.

In another embodiment of the present invention, the stent further comprises a therapeutic agent.

### Definition of Terms

Before proceeding it may be useful to define many of the terms used to describe the present invention. Words and terms of art used herein should be first defined as provided for in this specification, and then as needed as one skilled in the art would ordinarily define the terms.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Bioabsorbable: As used herein “bioabsorbable” refers to a material that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however all bioabsorbable polymers are considered biodegradable.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein as first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Compatible: As used herein “compatible” refers to a composition posing the optimum, or near optimum combination of physical, chemical, biological and drug release kinetic properties suitable for a controlled-release coating made in accordance with the teachings of the present invention. Physical characteristics include durability and elasticity/ductility, chemical characteristics include solubility and/or miscibility and biological characteristics include biocompatibility. The drug release kinetic should be either near zero-order or a combination of first and zero-order kinetics.

Delayed Release: As used herein “delayed release” refers to the release of bioactive agent(s) after a period of time and/or after an event or series of events.

Drug or Therapeutic agent: As used herein “drug” or “therapeutic agent” shall include any agent having a therapeutic effect in an animal. Exemplary, non limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARy), hypothemycin, nitric oxide, bisphosphonates, epidermal growth factor inhibitors, antibodies, proteasome inhibitors, antibiotics, anti-inflammatories, anti-sense nucleotides and transforming nucleic acids, cytostatic compounds, toxic compounds, anti-inflammatory compounds, chemotherapeutic agents, analgesics, antibiotics, protease inhibitors, statins, nucleic acids, polypeptides, and delivery vectors including recombinant micro-organisms, liposomes, the like.

### DETAILED DESCRIPTION OF THE INVENTION

The present invention provides bioabsorbable magnesium-reinforced stents which combine the radial strength and flexibility of metal stents with the controlled drug delivery properties of polymers. The radial strength of bioabsorbable polymer stents is lower than metal stents with comparable dimensions. Substantially increasing the thickness of bioabsorbable polymer stents to increase the radial strength will likewise increase the crossing profile and wall thickness, rendering the stent unsuitable for its intended purpose. Therefore, bioabsorbable polymer stents reinforced with bioabsorbable magnesium or magnesium alloys are provided.

Several methods are available for reinforcing bioabsorbable polymeric materials with bioabsorbable magnesium materials including but not limited to the use of bioabsorbable magnesium wire, magnesium fibers either wound around or within a polymeric stent or impregnated within a polymeric stent.

Magnesium and its alloys are biocompatible, bioabsorbable and easy to mechanically manipulate presenting an attractive solution for reinforcing bioabsorbable polymer stents. Radiological advantages of magnesium include compatibility with magnetic resonance imaging (MRI), magnetic resonance angiography and computed tomography (CT). Vascular stents comprising magnesium and its alloys are less thrombogenic than other bare metal stents. The biocompatibility of magnesium and its alloys stems from its relative non-toxicity to cells. Magnesium is abundant in tissues of animals and plants, specifically Mg is the fourth most abundant metal ion in cells, the most abundant free divalent ion and therefore is deeply and intrinsically woven into cellular metabolism. Magnesium-dependent enzymes appear in virtually every metabolic pathway is also used as a signaling molecule. Magnesium alloys which are bioabsorbable and suitable for reinforcing bioabsorbable polymer stents include alloys of magnesium with other metals including, but not limited to, aluminum and zinc. In one embodiment, the magnesium alloy comprises between about 1% and about 10% aluminum and between about 0.5% and about 5% zinc.

The magnesium alloys of the present invention include but are not limited to Sumitomo Electronic Industries (SEI, Osaka, Japan) magnesium alloys AZ31 (3% aluminum, 1% zinc and 96% magnesium) and AZ61 (6% aluminum, 1% zinc and 93% magnesium). The main features of the alloy include high tensile strength and responsive ductility. Tensile strength of typical AZ31 alloy is at least 280 MPa while that of AZ61 alloy is at least 330 MPa.

The present invention provides for bioabsorbable magnesium-reinforced polymeric stents. Bioabsorbable polymers suitable for forming the stents of the present invention include, but are not limited to, polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactone polymers, polytrimethylene carbonate, polyacrylates, polyethylene glycol, hydrogels, photo-curable hydrogels, terminal diols, and combinations thereof.

The stent architectures suitable for fabrication of the bioabsorbable magnesium-reinforced polymer stents of the present invention are not limited to the examples provided herein but can include coil stents, helical spiral stents, woven stents, individual ring stents, sequential ring stents, closed cell stents, open cell stents, laser cut tube stents, ratcheting stents, modular stents and the like. Additionally, bioabsorbable stents made according to the teachings of the present invention include stents adapted for deployment in any vessel or duct to maintain patency including, but not limited to vascular stents, stent grafts, biliary stents, esophageal stents, and stents of the trachea or large bronchi, ureters, and urethra.

In one embodiment of the bioabsorbable magnesium-reinforced stents of the present invention, the stents are manufactured by laser cutting stent tubes manufactured from magnesium metal coated with a bioabsorbable polymer. In one embodiment, magnesium wire, less than approximately 0.15 mm in outer diameter, is wound into a close pitch coil and encapsulated with a bioabsorbable polymer. A stent is then laser-cut from the encapsulated coil.

In another embodiment of the laser cut bioabsorbable magnesium-reinforced stents of the present invention, a magnesium wire, less than approximately 0.15 mm in outer diameter, is filament wound into a flat paddle shape, the wire is encapsulated with a bioabsorbable polymer and then sheets are cut from the paddle. The sheets are then wound around a mandrel, compressed and heated to form a tube and the tube is laser cut to form a stent.

In another embodiment of the laser cut bioabsorbable magnesium-reinforced stents of the present invention, short length small diameter magnesium fibers, between approximately 1 mm and 5 mm in length with an outer diameter less than approximately 0.15 mm, are extruded into thin sheets which result in the orientation of the fibers in the direction of the length of the sheet and the sheet is wrapped on a mandrel and compression molded to form a tube. Alternatively, small diameter short length magnesium wires are extruded into tubing with the fiber length oriented to the tubing length. In each configuration, tubes would be laser cut to form a stent. The stents formed in this manner have increased radial strength when the magnesium fibers are oriented to the length of the stent rather than the circumference of the stent.

In another embodiment of the bioabsorbable magnesium-reinforced stents of the present invention, the stents are ratcheting stents. In one embodiment of a ratcheted stent according to the present invention, the stent is formed from a flat sheet of magnesium filament wire wound into a flat paddle shape. The magnesium filament wire is coated with bioabsorbable polymer by spraying, solvent casting, or by thermally pressing sheets of bioabsorbable polymer onto the fiber according to methods known to persons skilled in the art.

In one embodiment of the ratcheting bioabsorbable magnesium-reinforced stents of the present invention, the magnesium fibers are secured with a tape material prior to cutting the fibers from the paddle mandrel to form a fiber panel. The fiber panel is then transferred to either a thermal press or a solvent/polymer casting apparatus. In thermal pressing, a bioabsorbable polymer encapsulates the magnesium wires via compression and thermal heating of the wire. In solvent/polymer casting, a bioabsorbable polymer dissolved in a solvent is impregnated into the magnesium wires and as the solvent evaporates, the polymer hardens around the encapsulated magnesium wire fibers.

In yet another embodiment of the ratcheting bioabsorbable magnesium-reinforced stents of the present invention, short length small diameter magnesium fibers, between approximately 1 mm and 5 mm in length with an outer diameter less than approximately 0.15 mm, are extruded into thin sheets which result in the orientation of the fibers in the direction of the length of the sheet. In another embodiment, small diameter short length magnesium wires are spread on thin bioabsorbable polymer sheets and the short fibers are thermally pressed into the sheet to reinforce the polymer. In another embodiment, short fibers are spread on a release film and the bioabsorbable polymer is solvent cast around the fibers. In another embodiment, bioabsorbable magnesium microspheres are used to reinforce a bioabsorbable polymer sheet and the sheet is then thermal pressed or solvent cast with the microspheres similar to the short fiber sheet forming process.

In another embodiment of the bioabsorbable magnesium-reinforced stents of the present invention, the stents are modular stents. Configurations for modular stents are based on forming a ring. In one embodiment of the modular bioabsorbable magnesium-reinforced stents of the present invention, small diameter biodegradable magnesium wire is wound into appropriately sized rings and the rings are sprayed with bioabsorbable polymer, and solvents applied to the wound ring to lock the fibers in place. After the polymer has dried the ring is formed into an architecture such as, but not limited to, a sinusoidal element, and the elements are bonded together to form a modular stent.

In another embodiment of the modular bioabsorbable magnesium-reinforced stents of the present invention, small diameter biodegradable magnesium wire is co-mingled with small diameter bioabsorbable magnesium filament and wound to form a ring. The co-mingled fibers of the ring and the wire are set by a method including, but not limited to, tack bonding or spraying with a bioabsorbable polymer. The ring is then compression molded to bond the bioabsorbable filaments to the magnesium wire. Post-molding, rings are formed into sinusoidal elements and the elements are bonded together to form a stent.

The magnesium-reinforcement of the bioabsorbable polymer stent according to the teachings of the present invention includes reinforcing all of the stent or some of the stent with bioabsorbable magnesium. In one embodiment of the present invention, only portions of the bioabsorbable polymer stent which bear the highest strain are reinforced with magnesium.

The bioabsorbable magnesium-reinforced polymers stents of the present invention are also useful for the delivery and controlled release of drugs. Drugs that are suitable for release from the stents of the present invention include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment of the present invention, the drug is covalently bonded to the bioabsorbable polymer. The covalently-bound drug is released in situ from the degrading polymer with the polymer degradation products thereby ensuring a controlled drug supply throughout the degradation course. The drug is released to the treatment site as the polymeric material is exposed through biodegradation.

In another embodiment of the present invention, the drug is contained within pores or reservoirs within the bioabsorbable polymer and is released in situ from the degrading polymer thereby ensuring a controlled drug supply throughout the degradation course.

The bioabsorable polymers of the present invention can be tuned to degrade at various rates by varying the monomer composition of the polymer

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent comprising a bioabsorbable magnesium-reinforced polymer.

2. The stent of claim 1 wherein said magnesium comprises magnesium and magnesium alloys.

3. The stent of claim 1 wherein said is a bioabsorbable polymer selected from the group consisting of polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations thereof.

4. The stent of claim 2 wherein said magnesium alloy comprises an alloy of magnesium, aluminum and zinc.

5. The stent of claim 1 wherein said stent is a vascular stent.

6. The stent of claim 1 wherein said stent is selected from the group consisting of woven stents, individual ring stents, sequential ring stents, closed cell stents, open cell stents, laser cut tube stents, ratcheting stents, and modular stents.

7. The stent of claim 1 wherein said stent is a helical spiral vascular stent.

8. The stent of claim 1 wherein said stent further comprises a therapeutic agent.
